(19) United States
(12) Patent Application Publication
(10) Pub. No.: US 2024/0375995 A1
(43) Pub. Date: Nov. 14, 2024

(54) COATED SUBSTRATES WITH EXTENDED SHELF LIFE

(71) Applicant: Trajan Scientific Australia Pty Ltd, Ringwood, Victoria (AU)

(72) Inventor: Herbert Foo, Ringwood, Victoria (AU)

(21) Appl. No.: 18/690,602

(22) PCT Filed: Sep. 9, 2022
(86) PCT No.: PCT/AU2022/051091

§ 371 Date: Mar. 8, 2024

(30) Foreign Application Priority Data
Sep. 10, 2021 (AU) 2021902935

(51) Int. Cl. C03C 17/00; A61B 5/15; B01L 3/00; C03C 17/34; C09D 5/16; C09D 133/02; C09D 139/02; C09D 177/04

(52) U.S. Cl. CPC C03C 17/004; A61B 5/150061; A61B 5/150343; B01L 3/5082; C03C 17/3405; C09D 5/1668; C09D 133/02; C09D 139/02; C09D 177/04; B01L 2300/0838; B01L 2300/16; B01L 2400/0406; C03C 2218/112

(57) ABSTRACT

The present disclosure relates to coated substrates, capillaries and microfluidic channels which exhibit an extended shelf life compared to uncoated substrates. More specifically the disclosure relates to charged coatings at physiological pH which may prolong the shelf life of the substrate, capillary or microfluidic channel before being applied to biological fluids. Said shelf life is measured by the ability to provide a low fluid contact angle after an extended storage interval in ambient atmospheric conditions.

### CROSS-REFERENCE TO RELATED APPLICATIONS

This application claims the benefit of priority to Australian provisional application 2021902935, filed 10 Sep. 2021, the entire contents of which is incorporated by reference herein.

### FIELD OF THE DISCLOSURE

This disclosure generally relates to coated substrates, particularly as present in, for example, capillaries and microfluidic channels. The coated substrates possess an extended shelf-life compared to uncoated substrates. The substrate may be, for example, a glass capillary, and the coating may prolong the shelf life of the capillary and enhance its usefulness in the collection of biological fluids, for example, blood samples. This disclosure also relates to a coating that, in some forms, reduces the impact of adventitious carbon deposition on the internal surface of a capillary on capillary action.

### BACKGROUND OF THE DISCLOSURE

Patient-centric devices for at-home diagnostic testing are of increasing importance. Such devices should be easy to use for patients or end-users so that the diagnostic tools generate consistent and reliable measurements.

With a trend to smaller sample volumes, a number of microfluidic point of care diagnostic devices have been developed for a range of analytic purposes. Most such devices are relatively costly and complex to operate and, in relation to blood, while avoiding the need to transport blood samples for analysis in laboratory settings, still require operation by skilled medical staff. It would be advantageous to the delivery of healthcare if microfluidic blood samples could be collected accurately by relatively untrained personnel or even by patients themselves. Devices for self-testing of blood sugar level and home pregnancy kits are examples of successful products in which untrained individuals can perform diagnostic tests on themselves, in one case utilising finger-prick blood spots and in another urine collection.

A desirable requirement for the proper function of microfluidic point of care diagnostic devices is that they reliably and accurately fill a pre-determined volume of a capillary or channel in any orientation of the device.

In relation to the transfer of blood, Pyrex borosilicate glass capillaries are known to not transfer a prescribed volume of blood to a filter paper after the capillaries have been stored for about six months in an ambient atmosphere. This is known as an ‘aging’ event. The symptoms related to aging begin with a decrease in the rate of blood filling the capillary, followed by the blood being unable to fill the pre-determined volume of the capillary. Aging of the capillary likely corresponds to an increase in contact angle (CA) on the glass capillary's internal surface over a period of time. The increase in CA indicates that the affinity of the liquid to the glass surface decreases, or wettability of the substrate by the liquid decreases. A similar aging effect is known for metal, ceramic or polymeric based materials that are used in microfluidic devices.

The increase in the contact angle on the capillary or microfluidic channel prevents the capillary or channel from properly filling to a pre-determined volume. Proper filling of the capillary or channel to a pre-determined volume is a desirable performance metric of these devices, as they rely on an accurate sample volume. Therefore, the aging of the surface determines the shelf life of the capillary or channel.

It is possible to reverse the aging effects and regenerate the native surface and wettability of the capillary or channel by cleaning the surface. However, cleaning of internal surfaces remains a challenge, particularly in an end-user or home environment.

Alternatively, some devices require the user to fill the capillary by holding it in the negative plane. By inverting the capillary, the liquid flow is assisted by gravity, which compensates for the decreased wettability of the capillary surface. However, any increase in the complexity of the measurement, increases the potential for error on behalf of the user, potentially affecting the accuracy of results. Therefore, it is preferable that the capillary should fill with the required sample volume regardless of orientation. In other words, the capillary or channel should fill to a predetermined volume using capillary forces alone, and not require the assistance of gravity to force the blood into the capillary or channel.

The short shelf life of single-use components in at-home diagnostic devices, such as capillaries and microfluidic channels, leads to an increase cost and manufacturing complexity of these components.

Therefore, there is a need to improve the shelf life of components such as glass capillaries for use in patient-centric diagnostic tools.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE DISCLOSURE

This disclosure relates to coated substrates that exhibit an extended shelf life. Surprisingly the inventors have discovered that the aging of capillary or microfluidic channel surfaces may be influenced by the deposition of adventitious carbon (AC). Without wishing to be bound by theory the inventors propose that substrates coated with one or more layers independently comprising carboxylate functions, amine functions or combinations thereof, such as polyelectrolytes, can ameliorate the effects of adventitious carbon deposition on the wettability of the substrate, thus extending the shelf life of the substrates.

Adventitious carbon can be an issue in the field of surface analysis, particularly X-ray photo electron spectroscopy (XPS) where even extremely small quantities of adventitious carbon are visible on the surface. However, the relationship between aging of capillaries and the deposition of adventitious carbon has not yet been recognised.

In a first aspect of the present disclosure, there is provided a coated substrate comprising:
a substrate, said substrate comprising one or more of glass, ceramic, metal and polymer;
a coating, said coating comprising one or more coating layers, wherein the one or more coating layers coat at least one surface of the substrate, and wherein at least one of said one or more coating layers independently comprises carboxylate function, amine function or combinations thereof.

In some embodiments the at least one of said one or more coating layers comprises carboxylate function.

In some embodiments the at least one of said one or more coating layers comprises amine function.

In some embodiments the at least one of said one or more coating layers comprises polycarboxylate function.

In some embodiments the at least one of said one or more coating layers comprises polyamine function.

In some embodiments the at least one of said one or more coating layers independently consists of carboxylate function, amine function or combinations thereof.

In some embodiments, the contact angle of a biological fluid on the coated surface is less than 50°, or less than 40°, or less than 30°, or less than 25°.

In any and all embodiments of the present disclosure the biological fluid may be saliva, urine, tear fluid, mucus, blood, or any interstitial fluid. In preferred embodiments the biological fluid is blood.

In another embodiment the blood contact angle on the coated surface is less than 50°, or less than 40°, or less than 30°, or less than 25°.

In some embodiments the contact angle of a biological fluid on the coated surface does not increase to a value of more than 50°, or more than 40° after 6 months storage, preferably 9 months storage, more preferably 12 months storage, more preferably 18 months storage, most preferably 24 months storage at ambient conditions of temperature and pressure.

In some embodiments the contact angle of a biological fluid on the coated surface does not increase by more than 30° after 6 months storage under ambient conditions of temperature and pressure.

In some embodiments at least two of said one or more coating layers comprise carboxylate function.

In some embodiments at least two of said one or more coating layers comprise amine function.

In some embodiments at least two of said one or more coating layers comprise a combination of carboxylate and amine function.

In some embodiments the coating layers comprising carboxylate function comprise one or more polyelectrolytes and/or one or more poly(amino acids).

In some embodiments, the coating layers comprise one or more anionic polyelectrolytes and one or more cationic polyelectrolytes.

In some embodiments the coating layers comprise an outer layer comprising anionic polyelectrolyte.

In some embodiments the coating layers comprise an outer layer comprising a polyelectrolyte wherein the polyelectrolyte is anionic at physiological pH.

In some embodiments the coating layers comprise an outer layer comprising cationic polyelectrolyte.

In some embodiments the coating layers comprise an outer layer comprising a polyelectrolyte which is cationic at physiological pH.

In some embodiments the outer layer comprises poly(acrylic acid).

In some embodiments at least one of the coating layers comprises poly(allylamine).

In some embodiments the coating layers comprise one or more anionic poly(amino acids) and one or more cationic poly(amino acids).

In some embodiments the coating layers comprise an outer layer comprising anionic poly(amino acid).

In some embodiments the outer layer comprises poly(glutamic acid).

In some embodiments the outer layer comprises poly(lysine).

In some embodiments at least one of the coating layers comprises poly(arginine).

In some embodiments the coating layers comprise n+2 layers, comprising
a first layer and when present all odd number layers comprising anionic poly(electrolyte); and
a second layer and when present all even number layers comprising cationic poly(electrolyte),
wherein the first layer is in contact with the substrate; and
wherein n is an integer from 0 to 300.

In some embodiments the coating layers comprise n+2 layers, comprising
a first layer and when present all odd number layers comprising cationic poly(electrolyte); and
a second layer and when present all even number layers comprising anionic poly(electrolyte),
wherein the first layer is in contact with the substrate; and
wherein n is an integer from 0 to 300.

In some embodiments the coating layers comprise n+2 layers, comprising
a first layer and when present all odd number layers comprising anionic poly(amino acid); and
a second layer and when present all even number layers comprising cationic poly(amino acid),
wherein the first layer is in contact with the substrate; and
wherein n is an integer from 0 to 300.

In some embodiments the coating layers comprise n+2 layers, comprising
a first layer and when present all odd number layers comprising cationic poly(amino acid); and
a second layer and when present all even number layers comprising anionic poly(amino acid),
wherein the first layer is in contact with the substrate; and
wherein n is an integer from 0 to 300.

In some embodiments n is an integer between 2 and 100, preferably n is an integer between 2 and 20, even more preferably n is at least 10.

In some embodiments the thickness of the coating is from about 1 nm to about 450 nm, or from about 3 nm to about 100 nm, or from about 3 nm to about 30 nm.

In some embodiments the coating layers comprise four layers comprising a first layer and a third layer of cationic poly(electrolyte) and a second layer and a fourth layer of anionic poly(electrolyte), wherein the first layer is in contact with the substrate.

In some embodiments the coating layers comprise four layers comprising a first layer and a third layer of cationic poly(amino acid) and a second layer and a fourth layer of anionic poly(amino acid), wherein the first layer is in contact with the substrate.

In some embodiments the coating layers comprise four layers comprising a first layer and a third layer of anionic poly(electrolyte) and a second layer and a fourth layer of cationic poly(electrolyte), wherein the first layer is in contact with the substrate.

In some embodiments the coating layers comprise four layers comprising a first layer and a third layer of anionic poly(amino acid) and a second layer and a fourth layer of cationic poly(amino acid), wherein the first layer is in contact with the substrate.

In some embodiments the coating layers comprise at least twelve layers comprising a first layer and odd numbered layers of cationic poly(electrolyte) and a second layer and even numbered layers of anionic poly(electrolyte), wherein the first layer is in contact with the substrate.

In some embodiments the coating layers comprise at least twelve layers comprising a first layer and odd numbered layers of cationic poly(amino acid) and a second layer and even numbered layers of anionic poly(amino acid), wherein the first layer is in contact with the substrate.

In some embodiments the coating layers comprise at least twelve layers comprising a first layer and odd numbered layers of anionic poly(electrolyte) and a second layer and even numbered layers of cationic poly(electrolyte), wherein the first layer is in contact with the substrate.

In some embodiments the coating layers comprise four layers comprising a first layer and odd numbered layers of anionic poly(amino acid) and a second layer and even numbered layers of cationic poly(amino acid), wherein the first layer is in contact with the substrate.

In some embodiments, the coating layers are substantially free of functions that affect the fluorescence properties of reporter molecules with lanthanide-chelating-tags (LCTs) that are used in downstream fluorometric or immunofluorometric assays. Assays that require LCTs include hormone assays such as Growth hormone (GH), thyroid stimulating hormone (TSH), insulin, C peptide, as described in Kohek, M. B. F., Leme, C. R. M., Nakamura, I. T. et al. Effects of EDTA and Sodium Citrate on hormone measurements by fluorometric (FIA) and immunofluorometric (IFMA) methods. BMC Clin Pathol 2, 2 (2002).

In some embodiments, the outer coating layer is substantially free of functions that affect the fluorescence properties of reporting molecules used in downstream assays. In some embodiments, the coating layers are substantially free of functions that affect the fluorescence properties of SYPRO Orange a reporter molecule used in differential scanning fluorimetry for thermal shift assay (TSA) as described in Kroeger T, Frieg B, Zhang T, Hansen F K, Marmann A, Proksch P, et al. (2017) EDTA aggregates induce SYPRO orange-based fluorescence in thermal shift assay. PLoS ONE 12 (5).

In some embodiments the coating is substantially free of EDTA.

In some embodiments the coating is substantially free of small molecule chelates having a molecular weight less than about 300 g/mol.

In some embodiments the substrates comprises glass.

In some embodiments the glass comprises borosilicate glass, and wherein the ratio of the percentage of metal atoms to silicon and boron atoms in the borosilicate glass is at least 0.1.

In a second aspect of the present disclosure there is provided an article of manufacture comprising the coated substrate according to any one of the herein disclosed embodiments.

In some embodiments there is provided an article of the second aspect wherein one or more layers of the coated substrate is a spray coated layer.

In some embodiments there is provided an article of the second aspect, wherein the article comprises a capillary tube or a microfluidic channel.

In some embodiments the capillary tube or microfluidic channel comprises glass.

In some embodiments the coating coats an inner surface of the capillary tube or microfluidic channel.

In some embodiments the capillary tube has an aspect ratio (length: inner diameter) of from about 2:1 to about 150:1.

In some embodiments the capillary tube has an aspect ratio (length: cross sectional area) of from about 2:1 to about 191:1.

In some embodiments the capillary tube has an aspect ratio (length: inner diameter) of from about 2:1 to about 150:1 and the capillary has a minimum internal volume of 2.74 microliters and a maximum internal volume of 34.6 microliters.

In some embodiments the capillary tube has an aspect ratio (length: inner diameter) of from about 2:1 to about 150:1 and the capillary has a minimum internal diameter of about 358 micrometres and a maximum internal diameter of about 2000 micrometres.

In some embodiments the capillary tube has an aspect ratio (length: inner diameter) of from about 2:1 to about 150:1 and the capillary has a minimum length of about 4 millimetres and a maximum length of about 64 millimetres.

In some embodiments the capillary tube has an aspect ratio (length: cross sectional area) of from about 2:1 to about 191:1 and the capillary has a minimum internal volume of about 2.74 microliters and a maximum internal volume of about 34.6 microliters.

In some embodiments the capillary tube has an aspect ratio (length: cross sectional area) of from about 2:1 to about 191:1 and the capillary has a minimum cross sectional area of about 0.1 mm² and a maximum cross sectional area of about 3.1 mm².

In some embodiments the capillary tube has an aspect ratio (length: cross sectional area) of from about 2:1 to about 191:1 and the capillary has a minimum length of 4 millimetres and a maximum length of 64 millimetres.

In some embodiments the capillary tube fills with blood to a predetermined level when held in the vertical orientation to the blood sample and wherein the predetermined level is greater than 90%, preferably greater than 95% of the capillary volume under ambient conditions.

In some embodiments the capillary fills to the predetermined level in less than about 12 seconds, preferably less than about 11 seconds.

In some embodiments the internal area of the capillary ranges from 0.05 to 0.9 mm² and the length ranges from 4 to 64 mm.

In some embodiments the capillary fills to the predetermined level with an average rate of from about 0.2 μL/s to about 0.4 μL/s, preferably about 0.25 μL/s to about 0.45 μL/s. In some embodiments the capillary fills to the predetermined level with the rate of the increase in height of from about 0.4 mm/s to about 12 mm/s.

In some embodiments the capillary tube is stored for at least 6 months at ambient conditions of temperature and pressure prior filling with a biological fluid.

In any and all embodiments the biological fluid may be saliva, urine, tear fluid, mucus or blood. In preferred embodiments the biological fluid is blood.

In some embodiments the capillary tube is stored for at least 9 months, preferably at least 12 months at ambient conditions of temperature and pressure prior to filling with blood.

In a third aspect of the present disclosure there is provided a method of prolonging the shelf life of the coated substrate according to any one of the herein disclosed embodiments, whereby the coating inhibits the deposition of adventitious carbon on the substrate surface and/or ameliorates the effect of adventitious carbon adsorption on the contact angle of the biological fluid.

In a fourth aspect of the present disclosure there is provided a method of sampling a biological fluid comprising the step of drawing the biological fluid via capillary action into the capillary tube according to any one of the herein disclosed embodiments.

In some embodiments of the third and fourth aspects, the biological fluid may be saliva, urine, tear fluid or blood. In preferred embodiments the fluid is blood.

In a fifth aspect of the present disclosure there is provided use of the capillary tube according to any one of the herein disclosed embodiments to transfer a pre-determined volume of a sample of a biological fluid.

In some embodiments of the fifth aspect the biological fluid is blood.

In some embodiments the capillary tube is used with a microsampling device.

In some embodiments the capillary tube is used with a blood microsampling device.

In a sixth aspect of the present disclosure there is provided a method of manufacturing a capillary tube or microfluidic channel according to any one of the herein disclosed embodiments comprising the step of coating at least one surface of the capillary tube or microfluidic channel with at least one layer independently comprising carboxylate function, amine function or combinations thereof.

As used herein, except where the context requires otherwise, the term “comprise” and variations of the term, such as “comprising”, “comprises” and “comprised”, are not intended to exclude further additives, components, integers or steps.

It will be understood that where an embodiment is defined as “comprising”, the narrower position of “consists essentially of” and “consists of” are also disclosed. The term “consists essentially of” or variations such as “consisting essentially of” denotes that the embodiment includes all listed components or steps and may include other non-listed components or steps that do not materially affect the basic properties or function of the embodiment. The term “consists of” or variations such as “consisting of” is intended to denote that the embodiment is defined to exclude further additives, components or steps.

Further aspects of the present disclosure and further embodiments of the aspects described in the preceding paragraphs will become apparent from the following description, given by way of example and with reference to the accompanying drawings.

### BRIEF DESCRIPTION OF THE DRAWINGS

FIG. 1: A diagram showing a proposed aging mechanism of a glass surface due to adventitious carbon adsorption and the protection mechanism via the charge of the glass substrate with and without coating.

FIG. 2: Drawing showing the different chemical states of oxygen on a silicate glass surface, where M is the ion.

FIG. 3: Graph showing the percentage fill (% fill) of blood of N51a borosilicate glass, borosilicate glass (Vitrex) and borosilcate glass (Pyrex) before and after 2 and 3 days accelerated aging. The error bars are 2 standard error from the mean. Individual standard deviations are used to calculate the intervals.

FIG. 4: Graph showing the blood contact angle (BCA), High-resolution X-ray Photoelectron spectroscopy (HR XPS) measurement of Carbon atomic %, and the glass modifier ratio of heat-treated glass coverslips in Example 2. Graph legend: Treatment group A=Heat treated only, B=Stored organic+nitrogen environment for<1 day, C=Stored organic+nitrogen environment for 4-5 day, D=Stored organic+nitrogen environment for 22 day, E=Treated with water vapour, F=Treated with water plasma for 30 sec, G=Treated with water plasma for 5 mins. The error bars are 1 standard error from the mean.

FIG. 5: Graph showing the multiple linear regression analysis of the mean value of At % of C, % NBO+O²—and At % modifier/At % (Si+B).

FIG. 6: Graph showing the change in blood contact angle (ΔBCA=Median BCA after 2 days to accelerate aging minus Median BCA before 2 days to accelerate aging) on uncoated Trajan series 1 borosilicate coverslip and coverslips with different coatings.

FIG. 7: Graph showing blood contact angle measurements of layer-by-layer coated Trajan series 1 borosilicate glass coverslips when freshly prepared and after two days accelerated aging. The error bars are 2 standard error from the mean. Individual standard deviations are used to calculate the intervals.

FIG. 8: Graph showing change in blood contact angle (ΔBCA=Median BCA after 2 days to accelerate aging minus Median BCA before 2 days to accelerate aging) on uncoated soda glass and poly-lysine (Poly K) coated soda glass.

FIG. 9: Graph showing the percentage fill (% fill) of blood of uncoated and different types of dip coated Vitrex borosilicate glass capillaries before and after the accelerated aging experiment for 2 days. The error bars are 2 standard error from the mean. Individual standard deviations are used to calculate the intervals.

FIG. 10: Graph showing the percentage fill (% fill) of blood of uncoated and spray coated N51a borosilicate glass capillaries before and after the accelerated aging experiment for 2 days and 3 days. The error bars are 2 standard error from the mean. Individual standard deviations are used to calculate the intervals.

FIG. 11: Graph showing the percentage fill (% fill) of capillary coated with an anionic coating vs time (month) stored in ambient condition.

### DETAILED DESCRIPTION OF THE EMBODIMENTS

It will be understood that the disclosure disclosed and defined in this specification extends to all alternative combinations of two or more of the individual features mentioned or evident from the text or drawings. All of these different combinations constitute various alternative aspects of the disclosure.

Reference will now be made in detail to certain embodiments of the disclosure. While the disclosure will be described in conjunction with the embodiments, it will be understood that the intention is not to limit the disclosure to those embodiments. On the contrary, the disclosure is intended to cover all alternatives, modifications, and equivalents, which may be included within the scope of the present disclosure as defined by the claims.

For purposes of interpreting this specification, terms used in the singular will also include the plural and vice versa. For example, “a” means one or more unless indicated otherwise.

The use of the term “about” includes and describes the value or parameter per se. For example, “about x” includes and describes “x” per se. In some embodiments, the term “about” when used in association with a measurement, or used to modify a value, a unit, a constant, or a range of values, refers to variations of up to ±5% and/or ±10%. For example, “about 60° C.” in some embodiments includes 54° C.-66° C. and/or 57° C.-63° C.

Unless defined otherwise, all technical and scientific terms used herein have the same meanings as commonly understood by one of ordinary skill in the art to which this disclosure belongs. Although any materials and methods similar or equivalent to those described herein can be used to practice or test the present disclosure, the preferred materials and methods are now described.

One skilled in the art will recognise many methods and materials similar or equivalent to those described herein, which could be used in the practice of the present disclosure. The present disclosure is in no way limited to the methods and materials described.

The present disclosure relates to coated substrates. In a preferred embodiment of the disclosure the coated substrate is a coated glass capillary. In further embodiments of the disclosure the coated substrate is a coated channel, such as a coated microfluidic channel.

The present disclosure may be described by reference to a capillary or channel. It is understood that these terms may be used interchangeably.

The term “measurement” used in the specification should be construed in its broadest sense, including quantitative and qualitative measurement. Unless otherwise stated it should be understood that measurements are conducted at ambient conditions, i.e room temperature and pressure.

The terms Pyrex and N51a refer to different types of borosilicate glass with defined compositions known to the person skilled in the art. For example, the composition for N51a glass can be found in Schaut RA et al. Historical Review of Glasses Used for Parenteral Packaging. PDA J Pharm Sci Technol. 2017 July-August; 71. Similarly, Pyrex borosilicate glass refers to glass of a known composition, for example as described in Smedskjaer, M. M., Youngman, R. E. & Mauro, J. C. Principles of Pyrex® glass chemistry: structure-property relationships. Appl. Phys. A 116, 491-504 (2014).

The term “wettability” used in the specification refers to the ability of the surface to be wet by a particular liquid. The wettability of the surface is measured by measuring the contact angle, or the surface energy. The term “surface energy” refers to the excess free energy present at the surface compared to the bulk. Surface energy is typically measured by measuring the contact angle of liquids with different polarities (Extended Fowkes Model), however numerous other methods for measuring surface energy are well known in the literature.

The term “adventitious carbon” used in the specification refers to organic residues containing carbon which may adsorb on a substrate when the substrate is exposed to said residues, for example residues resulting from trace organic compounds present in the air.

### Capillary Rise

Capillary rise, h, the height that liquid with flow under only capillary forces is dictated by the below equation

$$h = \frac{2 γ cos θ}{ρ g r}$$

where γ is the surface tension of the liquid, θ is the contact angle of the liquid and the solid surface, ρ is the density of the liquid and ris the radius of the capillary and g is gravity.

It follows, from the above equation that as the contact angle increases, ie. the liquid is less able to wet the capillary and that the height that the liquid rises inside the capillary decreases.

For the device to function correctly, it is a requirement that the capillary fills to a pre-determined volume so that a known volume of, for example, blood is used for the diagnostic testing. The capillary should be easy for the end-user to fill, and not require that the capillary be held in a specific orientation to fill correctly. Therefore the capillary should desirably be able to fill from any orientation, for example from a vertical orientation. It will be understood that if the capillary is able to fill to a pre-determined volume in a vertical orientation, that is held at 90° from horizontal, the capillary is filling under capillary action without the assistance of gravity, and that the capillary will be able to fill at any orientation in the positive plane. In other words, if the capillary is able to fill in a vertical position, the capillary will also be able to fill at an orientation of for example 45° or 60° or 80° or 85° or 88° or 89° from horizontal.

It is desirable that the capillary is able to fill completely and/or fill to a pre-determined volume such that a reproducible aliquot of sample is transferred into the capillary. In one embodiment of the present disclosure a capillary that has been stored under ambient conditions (i.e on the shelf) for at least 6 months, preferably at least 9 months, more preferably at least 12 months, is able to fill to greater than 90% of the capillary volume. Preferably, the stored capillary is greater than 91%, or greater than 92% or greater than 93%, or greater than 94%, or greater than 95%, or greater than 96%, or greater than 97%, or greater than 98%, or greater than 99% or to 100%. Alternatively, the stored capillary of the present disclosure is able to fill to 90% or 91%, or 92% or 93%, or 94%, or 95%, or 96%, or 97%, or 98%, or 99% or to 100% of the capillary volume.

Alternatively or additionally in another embodiment of the disclosure a capillary that has been stored under stress conditions for 2 days (at 60° C. in a sealed poly-foil bag) is able to fill to greater than 90% of a target capillary volume. Preferably, the stored capillary is able to fill to greater than 91%, or greater than 92% or greater than 93%, or greater than 94%, or greater than or equal to 95%, or greater than or equal to 96%, or greater than or equal to 97%, or greater than or equal to 98%, or greater than or equal to 99% or to 100% of the capillary volume. More preferably a capillary that has been stored under stress conditions for 3 days (at 60° C. in a plastic bag) is able to fill to greater than 90% of the capillary volume. More preferably a capillary that has been stored under stress conditions for 3 days (at 60° C. in a plastic bag) is able to fill to greater than 91%, or greater than 92% or greater than 93%, or greater than 94%, or greater than or equal to 95%, or greater than or equal to 96%, or greater than or equal to 97%, or greater than or equal to 98%, or greater than or equal to 99% or to 100% of the capillary volume.

### Lucas-Washburn Equation and Capillary Dimensions

The Lucas-Washburn equation describes the relationship between the length L of a capillary that will fill, and the surface tension γ, dynamic viscosity η of the liquid and the capillary radius r and the contact angle θ of the liquid and capillary surface according to the relationship

$$L = \sqrt{\frac{γ rt cos ( θ )}{2 η}}$$

The dimensions of the capillary may vary as long as the internal diameter, length and wettability of the capillary are such that the capillary will fill to a predetermined level. In other words, the maximum length of a capillary of a given radius r which will fill is governed by the Lucas-Washburn equation. For example when the substrates of the present disclosure are glass capillaries, and the liquid used is blood, the capillary may be 15 mm in length with an internal diameter 0.75 mm. Alternatively the capillary may be 30 mm in length with an internal diameter of 0.36 mm. In a further embodiment of the disclosure the capillary may have a length between 15 mm and 30 mm and a corresponding radius less than or equal to the maximum radius determined by the Lucas-Washburn equation.

In some embodiments of the present disclosure the aspect ratio of the capillary, defined as the ratio of length to inner diameter may be about 2:1. In another embodiment of the disclosure, the aspect ratio of the capillary may be about 250:3. The aspect ratio of the capillary may be 2:1. The aspect ratio of the capillary may be 250:3. The aspect ratio of the capillary may be from 2:1 to 150:1 or any aspect ratio falling within that range, for example, but not limited to 25:1, 30:1, 40:1, 50:1, 100:1, 150:1. The aspect ratio of the capillary may be from about 15:1 to about 150:1 or any aspect ratio falling within that range, for example, but not limited to about 25:1, about 30:1, about 40:1, about 50:1, about 100:1, about 150:1.

Additionally or alternatively, in some embodiments the capillary tube has an aspect ratio defined as the ratio of the length to cross section area may be about 2:1. In another aspect of the disclosure the aspect ratio may be about 191:1. In another embodiment of the disclosure, the aspect ratio of the capillary may be about 191:1. The aspect ratio of the capillary may be 2:1. The aspect ratio of the capillary may be 191:1. The aspect ratio of the capillary may be from 2:1 to 191:1 or any aspect ratio falling within that range, for example, but not limited to 25:1, 30:1, 40:1, 50:1, 100:1, 150:1. The aspect ratio of the capillary may be from about 2:1 to about 191:1 or any aspect ratio falling within that range, for example, but not limited to about 25:1, about 30:1, about 40:1, about 50:1, about 100:1, about 150:1, about 191:1.

In some embodiments of the present disclosure the capillary has an internal volume of between 2.7 microliters and 35 microliters or any volume falling within that range. For example, the capillary has an internal volume of about 3 microliters, or about 3.5 microliters or about 4 microliters or about 5 microliters, or about 10 microliters, or about 15 microliters, or about 20 microliters, or about 25 microliters, or about 30 microliters, or about 35 microliters. In some embodiments of the present disclosure, the capillary has an internal volume of about 2.7 microliters. In some embodiments of the present disclosure the capillary has an internal volume of 5 microliters. In some embodiments of the present disclosure the capillary has an internal volume of 20 microliters. In some embodiments of the present disclosure the capillary has an internal volume of 35 microliters. In some embodiments of the present disclosure the capillary has an internal volume of about 5 microliters. In some embodiments of the present disclosure the capillary has an internal volume of about 35 microliters.

In some embodiments of the present disclosure the capillary has a minimum internal diameter of about 358 micrometres. In some embodiments of the present disclosure, the capillary has a maximum internal diameter of about 2000 micrometres.

The substrate, capillary or channel may comprise glass, for example borosilicate glass or soda-lime glass. The substrate, capillary or channel may comprise silicone. The substrate, capillary or channel may comprise a ceramic, metal or polymer based material.

Useful ceramic materials for manufacturing substrates, capillaries and channels include but are not limited to alumina, polycrystalline alumina, zirconia toughened alumina, zirconia and derivatives thereof, for example Yttria fully stabilised zirconia (ZDY), Magnesia partially stabilised zirconia (TTZ), Yttria partially stabilised Zirconia (YTZP), sapphire, silicon, ruby, silicon carbide (SiC) and derivatives thereof, for example direct-sintered SiC, reaction bonded SiC, nitride bonded SiC, Mullite.

Polymer based materials useful for substrates, capillaries, channels and microfluidic devices are PDMS, Bisphenol A Novolac epoxy (SU-8), polycyclo-olefin (PCO), polycarbonate (PC), Polypropylene (PP), Polyether ether ketone (PEEK), polytetrafluoroethylene (PTFE) and polystyrene (PS), photocurable soft perfluoropolyethers (PFPE), polymethylmethacrylate (PMMA), polyimide, perfluoroalkoxy (Teflon PFA), fluorinated ethylene propylene (Teflon FEP). Biodegradable polymers included polycaprolactone, poly (lactide-co-glycolkide) and polyglycolic acid (PGA) are also suitable for manufacturing substrates and microfluidic devices.

Useful metals for manufacturing substrates, capillaries, channels and microfluidic devices include titanium, aluminium, silver, copper, gold, platinum, palladium, alloys thereof, and stainless steel.

In embodiments the substrates, capillaries, channels or microfluidic devices comprise a metal coating. The metal coating may be used to promote conductivity. The metal used for coating may be a high-conductivity metal, for example gold, platinum and palladium.

In embodiments the substrate is pre-coated with a high conductivity metal.

### Product Shelf-Life

Product shelf-life is the finite time a product can be stored after manufacture under specified packaged and environmental conditions and still maintain the required performance criteria.

The shelf life for the herein disclosed coated substrates is tested in real time by storing the substrates in a plastic bag under ambient conditions. The required performance criteria of the coated glass capillaries is that the capillary fills to a predetermined volume when held vertically in contact with the liquid. Preferably the capillary fills to a predetermined volume within 12 seconds.

Accelerated shelf-life testing is performed by storing the coated substrate at elevated temperatures. The accelerated study is based on the relationship between temperature and reaction rate, where an increase in temperature increases the reaction rate.

Heat treatment for 2 days at 60° C., corresponds to a real-time storage of about 6 to about 9 months. Heat treatment at 3 days at 60° C. corresponds to a real-time storage of about 12 to about 18 months.

The contact angle is defined as the angle made by a droplet of liquid placed on top of a surface in air. In the context of the present disclosure the liquids of interest are biological fluids including saliva, tear fluid, urine, and blood. Each biological fluid will have a different contact angle depend on the interactions between the components in the biological fluid, in particular surface active components, such as proteins.

Notwithstanding the different compositions and presence of naturally occurring surface active components within different biological fluids, generally biological fluids are polar liquids. Therefore, it is understood that although the magnitude of the effect of adventitious carbon adsorption at the substrate surface on the contact angle of the biological fluid may differ from fluid to fluid, a general principal applies that change in surface energy of the substrate due to AC adsorption will increase the contact angle for all polar biological fluids on hydrophilic substrates.

When the liquid is blood, the contact angle of the blood on the substrate surface is referred to as the blood contact angle (BCA).

In the context of the present disclosure the surface is a glass, ceramic or polymer substrate, either uncoated or coated with at least one layer independently comprising carboxylate functions, amine functions or combinations thereof, such as one or more layers of polyanionic or polycationic coating. The biological fluid is considered to wet the surface when the contact angle is less than 90°. Preferably the contact is angle is less than 50°.

The blood contact angle on a freshly cleaned glass surface is typically 10° or less. However, after approximately six months of aging, or 2 days of accelerated aging the blood contact angle on an uncoated glass surface is about 50°.

The blood contact angle on a freshly prepared surface coated with at least one layer independently comprising carboxylate function, amine function or combinations thereof may be higher than the contact angle on the freshly prepared uncoated surface. The blood contact angle on the freshly prepared coated surface of the present disclosure may be between about 10° and about 40°

The blood contact angle on a freshly prepared surface coated with at least one layer comprising ammonium function may be higher than the contact angle on the freshly prepared uncoated surface. The blood contact angle on the freshly prepared coated surface of the present disclosure may be between about 10° and about 40°.

Even though there is an initial slight increase in contact angle of a freshly coated surface compared to a freshly prepared uncoated surface, the contact angle of the biological fluid, in particular blood, on the coated surface remains below 50° after 6 months storage.

In some embodiments the blood contact angle on the coated surface is about 10°, or about 15°, or about 20°, or about 25°, or about 30°, or about 40°. In some embodiments the blood contact angle on the coated surface when freshly prepared is less than 30°, or less than 25°.

In contrast to the uncoated surfaces, the inventors have found that the blood contact angle on a glass surface coated with at least one layer independently comprising carboxylate function, amine function or combinations thereof, preferably comprising a terminal layer which is polyanionic or polycationic at physiological pH, remains below 40° even after 2 days and/or 3 days of accelerated aging.

Preferably the blood contact angle on the coated surface remains below 50°, below 40°, preferably less than 30°, more preferably less than 25° after 2 days of accelerated aging. In another embodiment the blood contact angle on the coated surface remains below 50°, preferably less than 40°, more preferably less than 30° after 3 days of accelerated aging. In another embodiment the blood contact angle on the coated surface remains below 50°, preferably less than 40°, more preferably less than 30° after 6 months of aging in real-time. In another embodiment the blood contact angle on the coated surface remains below 50°, preferably less than 40°, more preferably less than 30° after 9 months of aging in real-time. In another embodiment the blood contact angle on the coated surface remains below 50°, preferably less than 40°, more preferably less than 30° after 12 months of aging in real-time. In another embodiment the blood contact angle on the coated surface remains below 50°, preferably less than 40°, more preferably less than 30° after 24 months of aging in real-time.

In any one of the embodiments of the present disclosure the increase in the blood contact angle after 2 days accelerated aging experiments is less than 40°, preferably less than 35°, preferably less than 30°, preferably less than 25°, preferably less than 20°.

In any one of the embodiments of the present disclosure the increase in the blood contact angle after 3 days accelerated aging experiments is less than 40°, preferably less than 35°, preferably less than 30°, preferably less than 25°, preferably less than 20°.

In any one of the embodiments of the present disclosure the increase in the blood contact angle after 2 days accelerating aging experiments is less than the increase in the blood contact angle compared to an uncoated glass surface. In any one of the embodiments of the present disclosure the increase in the blood contact angle after 3 days accelerating aging experiments is less than the increase in the blood contact angle compared to an uncoated glass surface.

In any one or more of the embodiments of the present disclosure it is preferred that a coated capillary, preferably a coated glass capillary, more preferably a coated borosilicate glass capillary will fill to a predetermined level with blood in about 12 seconds, preferably in less than 12 seconds. In an embodiment of the present disclosure, a coated glass capillary will fill to a predetermined level with blood in 12 seconds. In an embodiment of the present disclosure, a coated glass capillary will fill to a pre-determined level with blood in less than about ten seconds.

### Glass Modifier Ratio

A glass channel or capillary's surface chemistry can be controlled by selecting the composition of glass or performing physical or chemical surface treatment on the surface, such as etching (acidic or alkaline) followed by ion exchange and plasma treatment.

The inventors have surprisingly found that the glass surface chemistry may be usefully described as the glass modifier ratio. For a borosilicate glass, the glass modifier ratio may be defined as the ratio of the percentage of atomic metals to the percentage of atomic silicon+boron in the glass. The percentage of metal, silicon and boron atoms at the glass surface may be measured by a survey scan using XPS (X-ray photoelectron spectroscopy) or high resolution x-ray photoelectron spectroscopy (HR-XPS).

Alternatively, an estimate of the glass modifier ratio may also be calculated from the bulk composition of the glass. The bulk composition of the glass substrate may be known from the product specifications of the manufacturer, or may be measured by a suitable means known in the art, for example X-ray fluorescence, secondary ion mass spectroscopy, or using an electron probe microanalyzer (EPMA) or energy-dispersive X-ray spectroscopy (EDX).

The blood contact angle on the glass substrates with a high glass modifier ratio show decreased sensitivity towards the effects of adventitious carbon deposition. Glass substrates with a high glass modifier ratio show a smaller increase in blood contact angle compared to glass substrates with a similar amount of adventitious carbon but a lower glass modifier ratio. For example, in one embodiment of the present disclosure a glass substrate with a glass modifier ratio of about 0.55 or 0.6 with more than 18% adventitious carbon on the surface had a blood contact angle of less than 20°, compared to a glass substrate with a glass modifier ratio of less than 0.5 and 18% adventitious carbon on the surface with a blood contact angle of 40°.

In any one or more of the embodiments of the present disclosure, the glass modifier ratio may be greater than 0.1, preferably greater than 0.15, even more preferably greater than 0.2, most preferably greater than 0.3. The glass modifier ratio may be from about 0.1 to about 0.7. The glass modifier ratio may be about 0.1, about 0.15, about 0.2, about 0.25, about 0.3, about 0.35, about 0.45, about 0.5, about 0.55, about 0.6, about 0.65 or about 0.7.

Different formulations of glass may have different quantities of metals, and as such, different glass modifier ratios. A glass surface is a covalent network formed by silicon, oxygen and boron and is relatively stable. The metals, for example sodium, are held in the glass network by negatively charged oxygen, and are known as defect sites on the surface. Defect sites are useful for surface modification, however, they are also vulnerable to erosion which leads to degradation of the glass surface. Therefore one element of the present disclosure is to identify the optimal glass surface composition that may balance the competing requirements of wettability and robustness in the required application.

Without wishing to be bound by theory, it is proposed that both the control of surface charge and wettability are important to the increased shelf-life of the glass substrates of the present disclosure. Typically a clean glass surface has a slightly negative surface charge. The inventors have found that increasing the charge of the glass surface by coating substrates with at least one charged layer, preferably negatively charged layer, for example comprising carboxylate function or amine function or a combination thereof, preferably carboxylate function, preferably at least one polyelectrolyte layer, extends the shelf-life of said coated substrate, for example a coated capillary.

In some embodiments the coating may be permanently charged. In some embodiments the coating may be charged at physiological pH. The coating may be positively or negatively charged at physiological pH.

The surface charge of a solid substrate may be measured by any means standard in the art, for example by measuring the zeta potential.

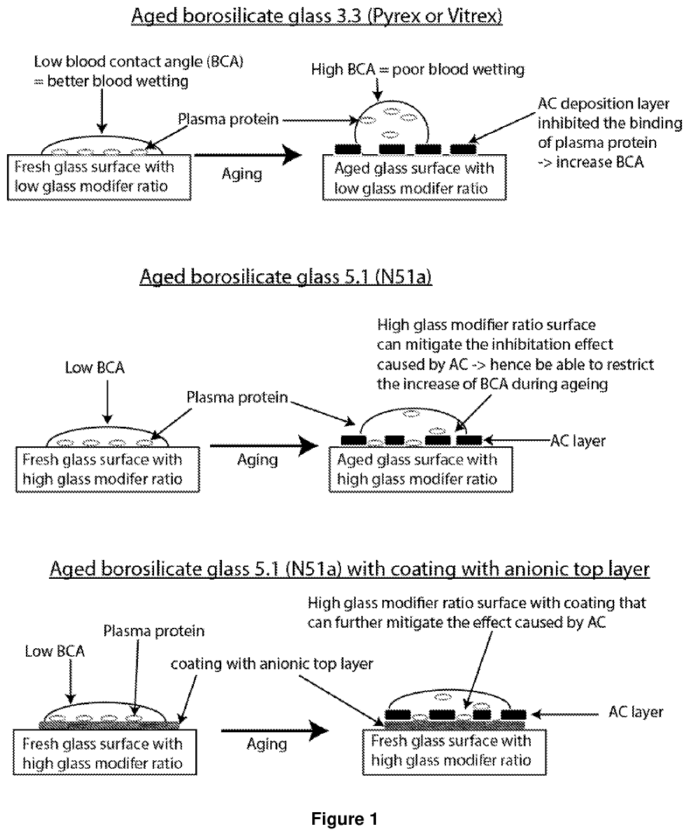

Without wishing to be bound by theory the inventors propose that the increased surface charge of the coated substrate increases the affinity of proteins present in the biological fluid to adsorb on the substrate, for example, plasma proteins in blood such as bovine albumin serum, lysozyme, fibrinogen and fibronectin, allowing the biological fluid, for example blood, to wet the substrate. A schematic of this is shown in FIG. 1. It can be appreciated that the adsorption due to increased affinity of any one of these plasma proteins to the positively or negatively charged surface can increase the wettability of substrate by blood. A similar relationship between proteins present in other biological fluids is expected.

For an uncoated substrate, adventitious carbon residues adsorbed on the surface of the substrate increase the contact angle of the biological fluid on the substrate, and thus decrease the substrate wettability. The inventors propose that although adventitious carbon resides may still be deposited on the coated substrates, the increased affinity of the proteins in the biological fluid with the charged surface compensates for any change in wettability caused by the adventitious carbon deposition. Without wishing to be bound by theory the inventors propose that by increasing the affinity of the proteins in the biological fluid to the substrate, the sensitivity of the biological fluid to the presence of adventitious carbon may be decreased. In this way, the substrate coated with a charged coating ameliorate the effects of adventitious carbon adsorption on the substrate. In some embodiments the changed coating may negate the effects of adventitious carbon adsorption on the wettability of the biological fluid. This proposed mechanism is shown in FIG. 1.

### Surface Active Oxygen

High energy oxygen atoms, which includes negatively charged atoms, atoms with electron vacancy or stressed bridging oxygen atoms, on the surface of the glass substrate may be referred to as active oxygen atoms. These oxygen atoms may act as potential binding sites for coatings and/or for plasma proteins in blood.

### Substrate Coating

The inventors have surprisingly found that increasing the negative surface charge of substrates by coating them with at least one layer comprising carboxylate function, amine function or combinations thereof are able to reduce the aging effect caused by adventitious carbon on the glass surface, thus extending the shelf-life of the glass capillary.

In some embodiments the substrates are coated by more than one layer, and at least one layer independently comprises carboxylate function, amine function or combinations thereof. In any and all embodiments of the present disclosure, the outer layer of the substrate coating, the layer in contact with the liquid, has a higher surface charge compared to uncoated substrate.

In some embodiments the substrates are coated by more than one layer, and at least one layer comprises carboxylate function. In preferred embodiments the first layer in contact with the substrate comprises cationic function, and the layer in contact with the liquid comprises anionic function. In any and all embodiments of the present disclosure, the outer layer of the substrate coating, the layer in contact with the liquid, has a higher negative surface charge compared to uncoated substrate.

In some embodiments the substrates are coated by more than one layer, and at least one layer comprises amine function. In preferred embodiments the first layer in contact with the substrate comprises anionic function, and the layer in contact with the liquid comprises cationic function. In any and all embodiments of the present disclosure, the outer layer of the substrate coating, the layer in contact with the liquid, has a higher positive surface charge compared to uncoated substrate.

The layer comprises a cationic function may be a polyelectrolyte or poly(amino acid). In some embodiments of the disclosure the cationic poly(amino acid) may be poly(arginine) (Poly R) or poly(allylamine) (PAH) or polylysine (poly K). The layer comprising an anionic function may be a polyelectrolyte or poly(amino acid). The anionic polyelectrolyte may be poly(acrylic acid). The anionic poly(amino acid) may be poly(glutamic acid) (Poly E).

In any and all of the embodiments of the disclosure the substrates, preferably glass surfaces, may be coated using, but not limited to, for example, dip coating, layer-by-layer coating, plasma assisted polymerisation, spray coating, drop casting, spin coating, chemical vapour deposition, plasma assisted vapour deposition and electrospray coating.

In some embodiments of the present disclosure the substrate or capillary may be coated by dip coating. Dip coating is the process of immersing a substrate in a tank containing the coating solution and removed from the immersion tank at a constant speed, allowing the liquid to drain from the surface. The thickness of the coating layer can be tuned by the concentration of the solution, the dwell time, the time the substrate remains immersed in the coating solution, and the speed in which the substrate is removed from the tank. Typically more rapid withdrawal (shorter drainage times) of the substrate results in thicker films and slower withdrawal creates a thinner film (longer drainage times). The coated substrate may be dried or baked at elevated temperatures and or under vacuum. The coating substrate may be cured or activated by ionizing radiation or plasma.

In some embodiments of the present disclosure the substrate or capillary may be coated by spray coating. In embodiments, spray coating is the process of creating an aerosol of the coating solution by mixing the solution with gas to form a spray, and the spray is directed onto the substrate's surface, e.g. capillary or slide.

In some embodiments of the present disclosure, at least some of the coating layers are deposited by spray coating. In some embodiments all of the coated layers are deposited by spray coating.

In some embodiments of the present disclosure, the substrate is coated using plasma assisted polymerisation as described in Kirby. G et.al Applied Materials & Interfaces 2017 9 (4).

In some embodiments, the coated substrates are coated using layer-by-layer deposition. Layer-by-layer (LbL) deposition is a surface coating technique where the surface is coated by depositing alternative layers of charged materials with wash steps in between. For example a four layer film may be deposited on a negatively charged surface by a washing step (W), followed by a polycation (+), followed by a washing step, followed by a polyanion (−), followed by a washing step, followed by a polycation, followed by a washing step, followed by a polyanion such that the process is depicted as W+W−W+W−W+W−, and the coating includes alternating cationic and anionic layers depicted as +−+−, or alternatively as (+−)₂. Each individual layer may have a defined thickness and the deposition process may be repeated to build up a multilayer coating of a desired thickness and surface charge.

In some embodiments of the present disclosure the layer-by-layer coating comprises an even number of layers and the charge of the first layer (i.e the layer in contact with the substrate) has the opposite charge to the final layer. In some embodiments the layer-by-layer coating comprises an odd number of layers and the charge of the first layer (i.e the layer in contact with the substrate) has the same charge as the final layer.

In some embodiments of the present disclosure the layer-by-layer coating may comprises a minimum number of coating layers of at least 2 layers, at least 4 layers, at least 5 layers, at least 6 layers, at least 7 layers, at least 8 layers, at least 9 layers, at least 10 layers, at least 11 layers, at least 12 layers, at least 13 layers, at least 14 layers, at least 15 layers, at least 16 layers, at least 17 layers, at least 18 layers, at least 19 layers, or at least 20 layers. In some embodiments the layer-by-layer coating may comprises a maximum number of layers of not more than 300 layers, not more than 250 layers, not more than 200 layers, not more than 150 layers not more than 100 layers, or not more than 50 layers. In some embodiments the coating layers may comprise from any one of these minimum number of layers to any one of these maximum number of layers, for example from at least 4 layers to at least 200 layers, from at least 10 layers to at least 150 layers, from at least 12 layers to at least 300 layers.

In some embodiments of the present disclosure the layer-by-layer coating comprises at least 4 layers. In some embodiments of the present disclosure the layer-by-layer coating comprises at least 4 layers, or at least 5, or at least 6 layers, or at least 7, or at least 8 layers, or at least 9 or at least 10 layers, or at least 11, or at least 12 layers, or at least 13, or at least 14, or at least 15, or at least 16, or at least 17, or at least 18, or at least 19, or at least 20 layers. In a preferred embodiment of the present disclosure the layer-by-layer coating comprises at least 12 layers.

In embodiments the thickness of each polyelectrolyte coating is about 1.5 nm.

In embodiments where the coated substrate is a capillary or channel, the total number of layers is chosen such that the coating does not appreciably change the volume of the capillary or channel. Preferably the maximum total thickness of the coating is about 450 nm, or about 400 nm, or about 350 nm, or about 300 nm, or about 250 nm, or about 200 nm, or about 150 nm, or about 100 nm.

In some embodiments of the present disclosure the layer-by-layer coating may comprises a minimum thickness of about 1 nm, about 1.5, about 2 nm, about 3 nm, about 4 nm, about 4.5 nm, about 5 nm, about 6 nm, about 7 nm, about 7.5 nm, about 8 nm, about 9 nm, about 10 nm, about 11 nm, about 12 nm, about 13.5 nm, about 15 nm, about 16.5 nm, about 18 nm, about 19.5 nm, about 21 nm, about 22.5 nm, about 24 nm, about 25.5 nm, about 27 nm, about 28.5 nm, or about 30 nm. In some embodiments the layer-by-layer coating may comprises a maximum thickness of about 450 nm, about 375 nm, about 300 nm, about 225 nm, about 150 nm, or about 75 nm. In some embodiments the coating layers may comprise from any one of these minimum thicknesses to any one of these maximum thickness, for example from about 6 nm to about 375 nm, from about 15 nm to about 225 nm, or from about 18 nm to about 450 nm.

In some embodiments the thickness of the coating on the coated substrate is from about from about 1 nm to about 450 nm, preferably from about 3 nm to about 100 nm, more preferably from about 3 nm to about 30 nm.

The layers are immobilised on the surface via predominantly electrostatic interactions but also hydrogen bonding, hydrophobic forces, acid-base interactions or even covalent bonding. Each individual deposition may be performed by, for example but not limited to spin coating, spray coating, adsorption, fluidics, drop-casting, dip coating or any other suitable means known in the art.

The term poly-ion refers to any multiply charged ion, including but not limited to polyelectrolytes, such as polycations, polyanions, polyampholytes, polyzwitterions or polysalts. The polyelectrolytes may be poly(amino acids) with a single type of amino acid as the monomer. The polyelectrolytes may be permanently charged, or may exhibit a net charge a physiological pH. For the purposes of the present disclosure the prefix ‘poly’ is understood to refer to molecule containing at least three repeating units.

In some embodiments of the present disclosure the prefix ‘poly’ refers to molecules containing from at least 10 to at least 100 repeating units. In some embodiments the polyions of the present invention refer to molecules containing at least 10, at least 20, at least 30, at least 40, at least 50, at least 60, at least 70, at least 80 at least 90 or at least 100 repeating units. In some embodiments, the prefix ‘poly’ refers to molecules containing more than 100 repeating units.

In some embodiments the molecular weight of the polyions of the present disclosure may range from 3 to 450 kDa. Alternatively in some embodiments the polyions may be referred to by the number average molecular weight. In some embodiments the number average molecular weight of the polyions may be between 3 and 450 kDa.

The number of layers deposited in a LbL coating controls the type of charge and the charge density on the surface.

Typically the polyions used are polyelectrolytes, or charged polymers, including but not limited to polycations such as poly(ethyleneimine) (PEI), poly(allylamine), poly(l-lysine), poly(dimethyldiallylammonium chloride), poly(allylamine hydrochloride), and chitosan (or chitosan derivatives), and polyanions such as poly(styrenesulphonate) (PSS), poly(vinylsulphonate), poly(acrylic acid) (PAA), poly(methacrylic acid) (PMA), and poly(anilinepropanesulphonic acid) (PAPS), polyallylamine (PAH), polyglutamic acid (Poly E), polyarginine (Poly R), polylysine (Poly K). In the context of the present disclosure, it is desirable that the polyelectrolyte maintains its charge (either positive or negative) across the relevant pH range of the biological fluid. For example when the biological fluid is blood, the pH is physiological pH. When the biological fluid is urine, the relevant pH range is from about 5 to about 8.

In preferred embodiments of the present disclosure the polycation is PEI or PAA. Preferably the polyanion is PAA or Poly E or poly R.

Layer-by-layer coatings may be characterised by analytical methods known in the art, for example but not limited to Zeta-Potential, QCMD (Quartz-crystal-microbalance) or Ellipsometry for example see: A. François, H. T. C. Foo, and T. M. Monro, “Polyelectrolyte Multilayers for Surface Functionalization: Advantages and Challenges,” in Advanced Photonics, OSA Technical Digest (online) (Optical Society of America, 2014)

### Coated Capillaries

In any and all embodiments of the present disclosure, the substrate may be a capillary, preferably a glass capillary, more preferably a borosilicate glass capillary.

Examples of coating methods which may be used to coat capillaries are, but are not limited to aspirating a drop of a solution containing a carboxylate function, amine function or combination thereof, inside a capillary as part of a layer-by-layer coating and drying it. The capillary may be coated via dip coating, spray coating, chemical vapour deposition, plasma assisted vapour deposition and electrospray coating.

In some of the embodiments of the present disclosure, the capillary is coated using a layer-by-layer deposition method where each layer is coated via dip coating. In some of the embodiments of the present disclosure, the capillary is coated using a layer-by-layer deposition method where each layer is coated via spray coating.

In some embodiments of the present disclosure the coated glass substrate comprises a capillary and is used with a blood sampling device, blood microsampling device and/or blood sampling protocol. Suitable devices are described in U.S. Pat. Nos. 9,606,032B2 and 20,182,35528A1. Kits useful for carrying out the present disclosure may comprise a blood sampling device, a coated capillary and/or instructions (e.g., printed instructions) for carrying out a method of blood sampling and/or analysis, optionally packaged together in a common package or container.

### Downstream Assays

The inventors have determined that coated substrates with a negative surface charge have an improved shelf-life. However, it is important to note, that as the collected biological fluid sample will be used in an assay after collection (also called downstream assay), the coating should not interfere with the assay results.

Assays may be for targeting trace amounts of inorganic ions, such as sodium, potassium, calcium magnesium, phosphorus and zinc that can be chelated with for example, EDTA. Coated capillaries with coatings that sequester the target positively charged inorganic ions may not be suitable for use for these types of assays. In some embodiments the coating is chosen such that at least the outmost layer of the coating does not sequester the target metal ions.

A negatively charged coating created by the deposition of salts, particularly sodium and potassium salts, from an aqueous solution may not be suitable for some assays. The increased salt concentration can affect the ionic strength of the aqueous solution which can alter the quantum yield of certain fluorescence molecules. As such, capillaries coated with a coating created by salt deposition from an aqueous solution may not be suitable for sample collection where the downstream assay requires a fluorescence molecule. In some embodiments the coating is not created by the deposition of salts.

Certain chelators are also known to affect the fluorescence properties of the reporter molecules such as the quantum yield and fluorescence lifetime. As such coatings that comprise or may leech strong chelators, such as EDTA, may not be suitable for use in down-stream assays that rely on fluorescence reporter molecules. In some embodiments the coating is chosen such that there is no substantial leeching of chelators from the coated substrate. In some embodiments the coating does not comprise strong chelators capable of interfering with fluorescent reporting molecules used in downstream assays such as the one using lanthanide chelate tagged reporter molecules used in downstream fluorometric or immunofluorometric assay for measuring hormones (e.g. Growth hormone (GH), thyroid stimulating hormone (TSH), insulin, C peptide, Sex hormone binding globulin (SHBG), total triiodothyronine (T3), total levothyroxine (T4), estradiol, testosterone, cortisol, and progesterone) and the one using SYPRO Orange, which is the reporter molecule used in differential scanning fluorimetry for thermal shift assay (TSA) that is used to measure changes in the thermal denaturation temperature and hence stability of a protein under varying conditions such as variations in drug concentration.

In one embodiment of the invention, the coating is chosen such that the shelf-life of the coated capillary is extended and the coating is compatible with the down-stream analysis intended for the collected biological sample.

### EXAMPLES

### Example 1: Measurement of Adventitious Carbon (AC) on Glass Surfaces

The internal surfaces of aged Pyrex borosilicate glass capillaries (from Vitrex) were analysed using time-of-flight secondary ion mass spectrometry (TOF-SIMS). In this analysis, capillaries were stored in either a plastic device, which was stored in a sealed polyfoil bag or an aluminium container which has a plastic internal coating. The containers were stored under ambient conditions (air atmosphere, room temperature and pressure) from three to more than twelve months. The internal surfaces of the stored capillaries were compared to the internal surface of a heat-treated capillary (control sample). The internal surface of the capillaries with longer storage time with plastic material were found to contain more organic residues compared to heat-treated capillaries. The organic residues were characterised as adventitious carbon using TOF-SIMS.

In a follow-up XPS analysis, the increase of adventitious carbon percentage from 11 to 38% in both survey and high resolution XPS scan was observed on borosilicate glass cover slips as a function of time (22 days). These cover slips were stored in a nitrogen purged environment for organic chemicals storage.

These experiments demonstrate that adventitious carbon deposits on the surfaces of glass substrates stored under ambient conditions, both in the presence or absence of plastic materials. The amount of adventitious carbon deposition was observed to increase with increased time of storage.

### Example 2: Measurement of the Glass Modifier Ratio

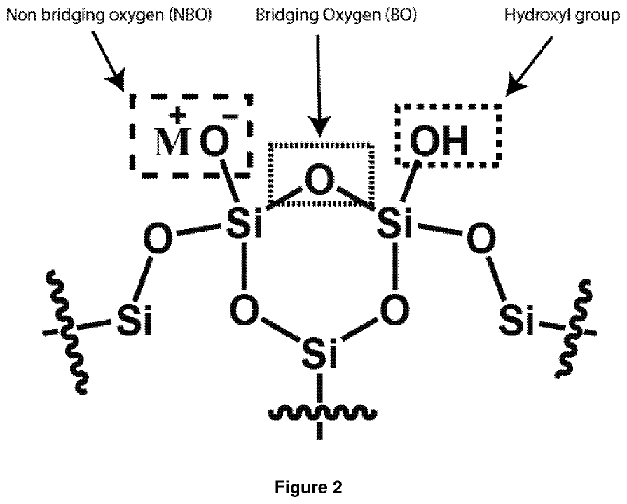

Different glass substrates have different bulk and surface compositions. One way to describe the surface composition of a glass is the glass modifier ratio. A schematic of the surface of an oxide glass is shown in FIG. 2.

The glass modifier ratio of oxide glass, which is the ratio of the atomic % of non-oxygen components that are not in the glass covalent structure (e.g. metal components with less than ˜20% in the overall glass composition) to the non-oxygen components that are in the glass covalent structure (e.g. silicon and boron), for different brand glass substrates were determined from XPS measurements. The glass substrates were borosilicate glass capillaries from Pyrex, Vitrex and N51a and borosilicate glass coverslip available as Trajan series 1 from Trajan Scientific Australia. For all capillary samples, the reported XPS data was measured from the capillaries internal surface which was exposed by cracking open the capillary before the XPS measurement. The glass modifier ratios for the different types of glass are shown in Table 1 below. The ‘HT’ descriptor following ‘Vitrex’ or ‘Trajan series 1’ in Table 1 means heat-treated, which is a treatment to remove adventitious carbon.

TABLE 1
The composition and the glass modifier ratio of different glass type

| | Pyrex | N51a | Vitrex | Trajan |
|---|---|---|---|---|
| | Internal | Internal | Internal | Series 1 |
| Atomic % | surface HT | surface HT | surface HT | surface HT |
| O | 64.55 | 61.88 | 63.88 | 67.41 |
| B | 4.06 | 4.23 | 4.94 | 1.16 |
| Na | 2.84 | 6.56 | 4.87 | 0.44 |
| K | 0.27 | 0.67 | 0.15 | 1.67 |
| Al | 1.01 | 2.50 | 0.84 | 2.14 |
| Si | 27.27 | 24.17 | 25.32 | 22.63 |
| Ca | 0.00 | 0.00 | 0.00 | 0.00 |
| Ba | 0.00 | 0.00 | 0.00 | 0.00 |
| Zn | 0.00 | 0.00 | 0.00 | 1.04 |
| Mg | 0.00 | 0.00 | 0.00 | 3.52 |
| Metal/B + Si | 0.13 | 0.34 | 0.19 | 0.37 |

### Example 3: Accelerated Aging Experiments

Capillaries aged under accelerated aging experiments (heat treatments) were compared to capillaries aged in real time under ambient conditions.

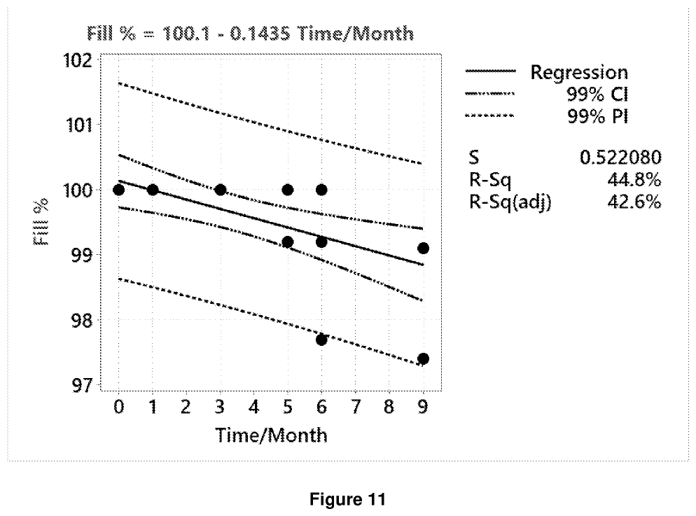

The percentage fill of blood N51a capillaries with anionic coating as a function of time stored in an ambient condition of 9 months are shown in FIG. 11. These results were used with prediction analytics based on the linear regression to predict the % fill after 9 months. The lower bound of the 99% prediction interval of percentage fill of blood of 6, 9, 12 and 18 months is 97.8, 97.3, 96.8 and 95.6%, respectively. The percentage fill of blood of the same type of capillary after 2 and 3 day heat treatment are 96.9±0.3 and 95.8±0.3%, respectively.

Based on these results, it was concluded that heat treatment for 2 days at 60° C., corresponds to a real time storage about 12 months and heat treatment at 3 days at 60° C. corresponds to a real time storage of about 18 months

### Example 4: Capillary Action Measurements of Aged Capillaries

The aging effect of borosilicate glass capillaries was accelerated by storing in sealed poly-foil bags under 60° C. for two to three days (corresponding to 6 months and 12 months shelf life respectively). It is proposed that during storage, adventitious carbon (AC) deposits on the capillary including the internal surface of the capillary. Following storage the capillary action of the aged capillaries was measured by the percentage fill (% fill) of a fixed length and ID capillary.

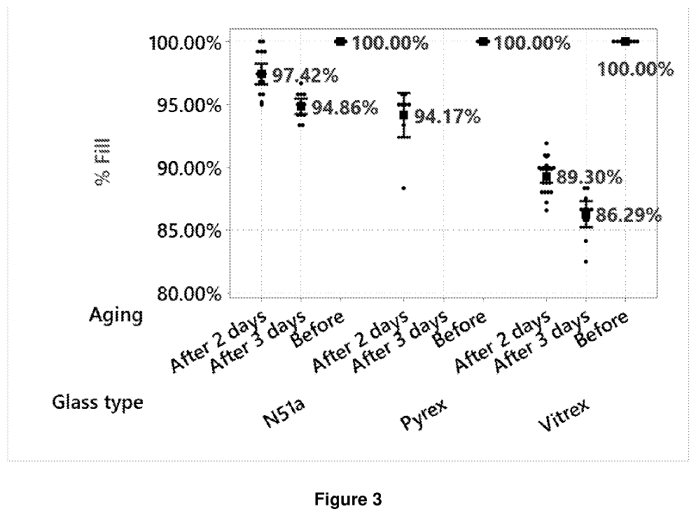

The results of this experiment are shown in FIG. 3 (borosilicate glass N51a, Vitrex and Pyrex capillaries). The results show that a reduction of % fill was observed after two days storage at 60° C. in a sealed poly-foil bag in any type of borosilicate glass without coating, and a further decrease of % fill was found after three days storage at 60° C. in a sealed poly-foil bag. The reduction in fill is attributed to AC adsorption on the glass surface. The observed reduction in % fill of the capillaries demonstrates the effect of AC on capillary action. It was found that the glass type with a higher glass modifier ratio, such as N51a (refer to Table 1), has less reduction of % fill. This result suggests that the glass modifier ratio plays a role in reducing the impact of AC on capillary action.

### Example 5: Surface Analysis of Glass Substrates Stored in Different Conditions and Relationship to Blood Contact Angle

To investigate the relationship between type of glass, adventitious carbon deposition and blood contact angle, glass cover slips were stored in different atmospheres under accelerated aging conditions and the blood contact angle, adventitious carbon deposition and glass modifier ratio of the substrates was measured.

Trajan series 1 coverslips, which is a type of borosilicate glass with a high glass modifier ratio, were treated in one of the following ways (a)-(g) to form a specific type of the glass surface chemistry condition. The treated substrate surfaces were analysed using XPS measurements on the treated samples to quantify the percentage carbon on the surface, the glass modifier ratio and the percentage of active oxygen on the samples. The blood contact angle was also measured on the treated substrate surfaces. The blood contact angle measures the wettability of the substrate.

The different treatments were:
(a) Heat treatment in an open-air furnace (Heat treated only (box furnace))
(b) Heat treatment in a nitrogen-rich environment and briefly stored in a nitrogen-purged glovebox for organic chemicals storage for less than 12 hours.
(c) Heat treatment in a nitrogen-rich environment and stored in a nitrogen-purged glovebox for organic chemicals storage for 4 to 5 days. The organic vapour in the glove box is a source of adventitious carbon.
(d) Heat treatment in a nitrogen-rich environment and stored in a nitrogen-purged glovebox for organic chemicals storage for 22 days. The organic vapour in the glove box is a source of adventitious carbon.
(e) Heat treatment in open-air furnace followed by water vapour treatment for 10 minutes in a vacuum plasma coating chamber.
(f) Heat treatment in open-air furnace followed by water vapour treatment for 10 minutes and then 30 seconds water plasma treatment in a vacuum plasma coating chamber. The source of adventitious carbon is mainly from the ambient environment.
(g) Heat treatment in open-air furnace followed by water vapour treatment for 10 minutes and then 5 minutes water plasma treatment in a vacuum plasma coating chamber. The source of adventitious carbon is mainly from the ambient environment.

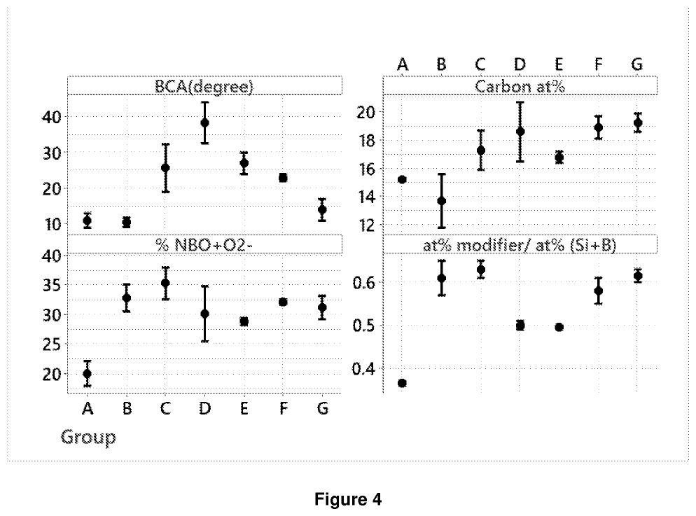

The results of these experiments are shown in FIG. 4. The blood contact angle measurements (top left) show that the heat-treated coverslips showed an increase in the blood contact angle when the glass coverslips are stored in a glove box with abundant organic vapour. The percentage of carbon atom (C) was measured on the surface using XPS (top right). The atomic % C on the glass surface increased across all forms of storage. However, there was not a direct correlation between the % C on the surface and the blood contact angle. This result also indicates that the AC is not the only controlling factor of BCA on the glass samples. If the AC were the only controlling factor, the graph of carbon atomic % in FIG. 4 (top left) would have a similar pattern as the BCA's graph (FIG. 4 top right).

Further surface characterisation of the % surface oxygen (both O²⁻-and non-bridging oxygen, and bridging oxygen) (bottom left) and the glass modifier ratio (bottom right) were measured using XPS. XPS analyses were performed using Kratos XPS fitted with a monochromatic aluminium X-ray source. Charged compensation was applied during each measurement. On each sample, three random spots were analysed by the XPS. From each spot, a survey scan and high resolution (HR) scans on silicon, oxygen, carbon and potassium were performed. Case XPS version 2.3.15 was used for the data analysis. The binding energy of every spectrum from each spot was calibrated by the C-H peak from its corresponding HR spectrum of carbon; this C-H peak was set to be 284.8 eV based on the literature value (Moulder, J. F. Handbook of X-ray Photoelectron Spectroscopy; Jill Chastain, Ed.; 1992, 1992). The same calibration process was repeated for every spot. Hence the binding energy (BE) of all spectra from each spot was calibrated based its local environment. The relative sensitivity factors (RSF) provided by Kratos analytical were used in the quantification of the data. Shirley background subtraction was performed for each peak used in the quantification.

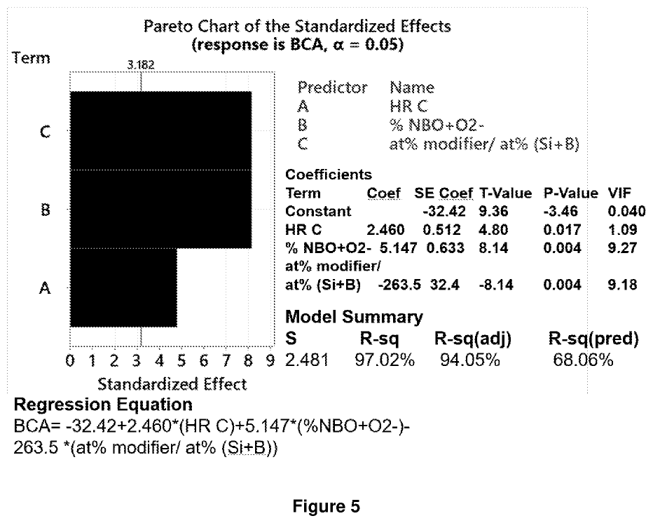

Multiple linear regression (MLR) analysis was performed on the results shown in FIG. 5 to understand the importance of different aspects of the glass surface chemistry on the aging affect and adsorption of adventitious carbon.

In silicate glass structure, every oxygen atom is in one of the four common chemical states with different binding energy (BE) that can be measured via high resolution XPS. The definition and BE of each of the four status are listed as follows; FIG. 2 shows the diagram of the structure of these chemical states. The chemical states are:
Bridging oxygen (BO): the oxygen between two silicon atoms, the BE is 532.2±0.2 eV;
Non-bridging oxygen (NBO): the oxygen that has one ionic bond to an metal atom and a covalent bond to a silicon atom, the BE is 531.2±0.2 eV,
Oxide ion (O²⁻): Oxide ion usually exists without directional bonding with another atom. It is stabilised by the positively charged atom in the glass matrix, the BE is 530.4±0.3 eV.
Hydroxyl groups (OH): Hydroxyl group on surface usually formed by chemisorption of water, the BE is 533.7±0.2 eV.

Multiple linear regression (MLR) was performed on the mean data of each of the sample types. In the MLR, the response was set to be BCA and the continuous predictors are carbon atomic percentage (C at %), glass modifier ratio and percentage of active oxygen (% of non-bridging oxygen (NBO) plus oxide ions (O²-). NBO and 0²⁻ are usually associated with the glass modifier compositions. This is corroborated by the variance inflation factor (VIF) value in FIG. 5.

The results of the multiple linear regression are shown in FIG. 5. The MLR model shows a good correlation coefficient (R² (Adjusted)=0.94). These results show that each of the factors: glass modifier ratio, adventitious carbon and surface oxygen positively contribute to the blood contact angle result. These results indicate that different glass substrates, and in particular different surface compositions have a significant contribution in controlling the effect caused by adventitious carbon.

This finding confirms the importance of the substrate surface chemistry on the deposition of adventitious carbon on the surface, and the aging effect of the substrate resulting in increased blood contact angle or decreased capillary action of aged substrates.

### Example 6: Coating Experiment on Glass Slides and Capillaries

Experiments to determine suitable charged coatings were performed using multiple layers of charged polymer coatings and ensuring the outermost or the liquid contact surface maintained positive or negative charge. The following two types of coatings were tested.

A glass coverslip substrate was coated using a radio frequency plasma-assisted polymerization technique with acrylic acid to form a PAA coating with carboxyl groups. The coating method is published by Kirby G et al, ACS Applied Materials & Interfaces 2017 9 (4), 3445-3454. Since the plasma energy has a negative relationship to the surface density of carboxyl groups, low energy plasma was used to promote the highest density of carboxyl groups.

A Trajan Series 1 borosilicate glass cover slip was coated with 4 layers polyelectrolyte coating with alternate layers of PAA and PAH, with PAA being the top layer and PAH being the layer in contact with the substrate. PAA and PAH were deposited using a layer-by-layer dip coating method. Since the glass surface is negatively charged, the deposition first started from 0.1 w/v % PAH solution, which is formed by dissolving PAH salt into a pH 4 phthalate buffer solution to ensure PAH polyelectrolyte is positively charged. Each polyelectrolyte layer deposition was formed by dipping the glass cover slip followed by 2 minutes incubation and then washed with de-ionised water. After the first layer deposition, 0.1 w/v % PAA solution was used to form the second layer. The PAA solution was made by dissolving PAA salt into pH 7 phosphate buffer. These process was repeated until 4 layers of polyelectrolyte was formed in PAH-PAA-PAH-PAA order. Alternatively the film may be described as (PAH-PAA)₂.

A Trajan Series 1 borosilicate glass coverslip was coated with 4 layers poly-(amino acid) coating with alternate layers of Poly R and Poly E with Poly E being the top layer and Poly E being the layer in contact with the substrate. Poly R and Poly E were deposited on the surface using a layer-by-layer dip coating method. Since the glass surface is negatively charged, the deposition first started from 0.1 w/v % Poly R solution, which is formed by dissolving Poly R salt into a pH 4 phthalate buffer solution to ensure Poly R polyelectrolyte is positively charged. Each polyelectrolyte layer deposition was formed by dipping the glass cover slip followed by 2 minutes incubation and then washed with DI water. After the first layer deposition, 0.1 w/v % Poly E solution was used to form the second layer. The PAA solution was made by dissolving Poly E salt into pH 7 phosphate buffer. These process were repeated until 4 layers of polyelectrolyte was formed in Poly-R-E-R-E order. Alternatively the film may be described as Poly (R-E)₂.

A Trajan Series 1 borosilicate glass coverslip was coated with a single layer of PAA or PAH. 0.1 w/v % PAA or PAH solution was used to form the single layer. The PAA solution was made by dissolving PAA salt into pH 7 phosphate buffer, and the PAH solution was made by dissolving PAH salt into pH 4 phthalate buffer solution. PAA or PAH single layer deposition was formed by dipping the glass cover slips followed by 2 minutes incubation and then washed with DI water. A Trajan Series 1 borosilicate glass cover slip was coated with a single layer of Poly-R or Poly-E. 0.1 w/v % Poly-R or Poly-E solution was used to form the single layer. The Poly-E solution was made by dissolving Poly-E salt into pH 7 phosphate buffer, and the Poly-R solution was made by dissolving Poly-R salt into pH 4 phthalate buffer solution. Poly-R or Poly-E single layer deposition was formed by dipping the glass cover slips followed by 2 minutes incubation and then washed with DI water.

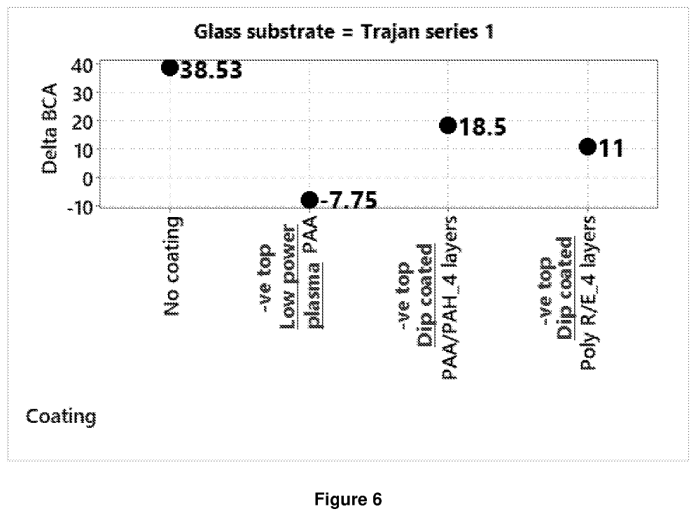

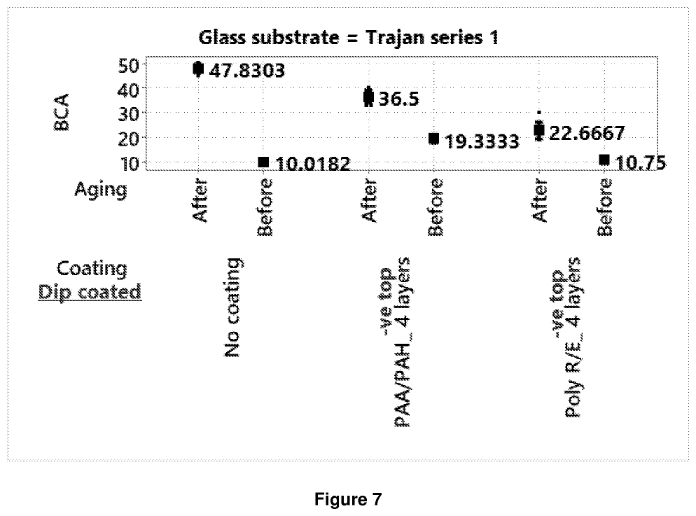

The change in blood contact angle (BCA) (ΔBCA=Median BCA after two days aging minus Median BCA before two days aging) on a coated glass slides was measured as an anti-AC effect performance metric. FIGS. 6 and 7 show the performance of each type of coating on Trajan series 1 borosilicate glass Coatings that showed a ΔBCA equal or less than 25° were considered to have a good performance. These experiments suggest that borosilicate glass surface coated with anionic coating can inhibit the increase of blood contact angle caused by AC deposition during aging.

### Example 7

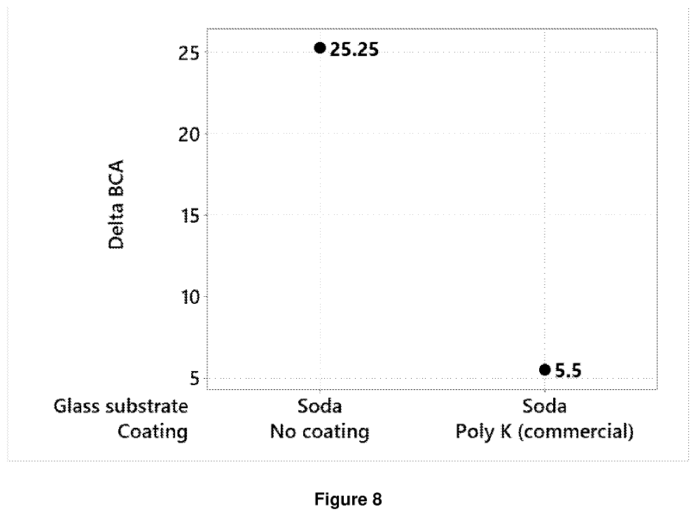

A soda glass slide was coated with a single layer of Poly K (poly-lysine) FIG. 8 shows the change in blood contact angle (BCA) (ΔBCA=Median BCA after two days aging minus Median BCA before two days aging) on soda glass slide and poly K coated soda glass slide (FIG. 8). Since the glass modifier ratio (0.4) in soda glass slide is higher than that (0.37) in borosilicate coverslip, the ABCA of uncoated soda glass (25) is smaller than that of uncoated borosilicate glass (40). FIG. 8 shows that on soda glass, the cationic Poly K coating can achieve ΔBCA below 25. This experiment suggests that the anti-AC effect can also be achieved by cationic coating if the glass substrate contains glass modifier ratio higher than 0.37. The glass modifier ratio was calculated from the composition reported in Maeng, J H et al. The effect of chemical treatment on the strength and transmittance of soda-lime cover glass for mobile. Int. J. Precis. Eng. Manuf. 15, 1779-1783 (2014).

Borosilicate glass (Vitrex) capillaries were coating using layer-by-layer deposition. Each layer was deposited by dip coating. The solution concentration, incubation time and the dip coating sequence with washing step for capillaries is the same as the one for slides (example 6). For capillaries, coating solution and water were delivered into the capillary via capillary action. The removal of liquid is also via capillary action that drives the liquid inside the bore to a clean tissue paper.

The following borosilicate glass (Vitrex) capillaries were prepared
(a) uncoated
(b) Coated with a single layer of PAA
(c) coated with a single layer of PAH
(d) coated with PAH-PAA-PAH
(e) coated with PAH-PAA-PAH-PAA, also written as (PAH-PAA)₂
(f) coated with a single layer of Poly E
(g) coated with a single layer of Poly R
(h) coated with Poly-R-E-R-E, also written as (poly-R-E)₂

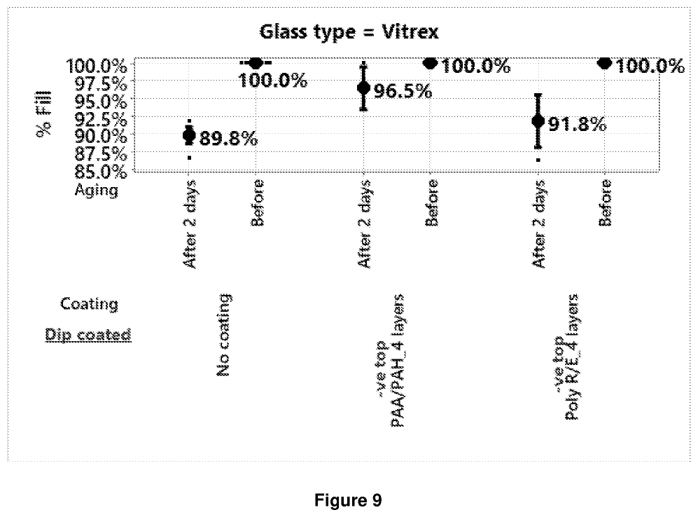

The fill volume of the capillaries was measured on a freshly prepared capillary, and on a capillary aged for 2 days at 60° C. in a sealed poly-foil bag. The results of the capillary fill experiments are shown in FIG. 9. The results from FIG. 9 show that LbL coated borosilicate glass capillaries with four layers and ending in an anionic layer, show the best resistance to the increase in BCA caused by aging after 2 days accelerated aging

Without wishing to be bound by theory, it is proposed that the performance of a 4 layer charged coating is improved relative to a single layer, potentially because of the increase in surface coverage and charge density. The increased negative charge density on the surface is proposed to increase the affinity of blood plasma proteins to adsorb on the substrate surface which facilitates the wetting of blood on the substrate. The increased affinity of the blood plasma proteins is proposed to compensate or ameliorate the increased surface energy caused by the deposition of adventitious carbon.

### Example 8

Borosilicate glass (N51a) capillaries were coating using layer-by-layer deposition. Each layer was deposited by spray coating method that utilises a vacuum pump to inhale a 3 μl, PAA or PAH solution droplet. The solution concentration of PAA or PAH was the same as Example 6. Before the spray coating process inside the capillary, the required number of droplets were dispensed on a glass slide. Deposition first started from PAH droplet followed by PAA droplet and the cycle repeated until the required number of layers were deposited into the capillary. No water washing is required in the spray coating process to speed up the deposition process for multiple layer deposition.

The following borosilicate glass (N51a) capillaries were prepared
(a) uncoated
(b) coated with (PAH-PAA)₂
(c) coated with (PAH-PAA)₆

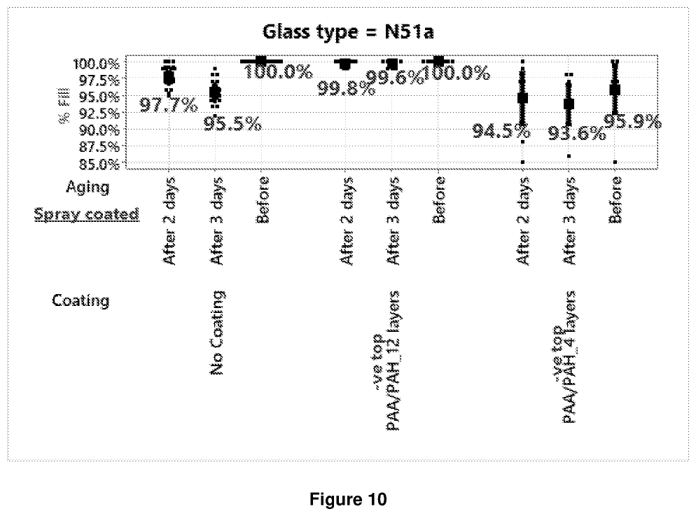

The fill volume of the capillaries was measured on a freshly prepared capillary, on a capillary aged for 2 days at 60° C. in a sealed poly-foil bag, and on a capillary aged for 3 days at 60° C. in a sealed poly-foil bag. The results of the capillary fill experiments are shown in FIG. 10. The results indicated that twelve layers polyelectrolyte ending in an anionic layer showed the best resistance to the increase in BCA on N51a glass surface caused by aging after 2 day and 3 day accelerated aging.

Without wishing to be bound by theory it is proposed that the performance of resistance to the increase in BCA is increased with an increased number of layers and ending with an anionic charged layer. It is potentially because of the increase in surface coverage and charge density. The increased negative charge density on the surface is proposed to increase the affinity of blood plasma proteins to adsorb on the substrate surface which facilitates the wetting of blood on the substrate. The increased affinity of the blood plasma proteins is proposed to compensate or ameliorate the increased surface energy caused by the deposition of adventitious carbon.

1. A coated substrate comprising:
a substrate, said substrate comprising one or more of glass, metal, ceramic and polymer;
a coating, said coating comprising one or more coating layers, wherein the one or more coating layers coat at least one surface of the substrate, and wherein at least one of said one or more coating layers independently comprises carboxylate function, amine function, polycarboxylate function, polyamine function or combinations thereof.

2. The coated substrate according to claim 1, wherein the contact angle of a biological fluid on the coated surface is less than 50°, or less than 40°, or less than 30°, or less than 25°.

3. The coated substrate according to claim 1, wherein the contact angle of a biological fluid on the coated surface does not increase by more than 30° after 6 months storage under ambient conditions of temperature and pressure.

4. The coated substrate according to claim 2, wherein the biological fluid comprises an interstitial fluid, saliva, urine, tear fluid, mucus or blood.

5-7. (canceled)

8. The coated substrate according to claim 1, wherein at least two of said one or more coating layers comprise carboxylate function, amine function, polycarboxylate function, polyamine function or combinations thereof.

9. The coated substrate according to claim 1, wherein the coating layers comprise one or more polyelectrolytes and/or one or more poly(amino acids).

10. The coated substrate according to claim 9, wherein the coating layers comprise one or more anionic polyelectrolytes and one or more cationic polyelectrolytes.

11-12. (canceled)

13. The coated substrate according to claim 9, wherein at least one of the coating layers comprises poly(allylamine).

14. The coated substrate according to claim 10, wherein the coating layers comprises one or more anionic poly(amino acids) and one or more cationic poly(amino acids).

15-18. (canceled)

19. The coated substrate according to claim 1, wherein the coating layers comprise n+2 layers, comprising
a first layer and when present all odd number layers comprising anionic poly(electrolyte) or an anionic poly(amino acid); and
a second layer and when present all even number layers comprising cationic poly(electrolyte) or a cationic poly(amino acid),
wherein the first layer is in contact with the substrate; and
wherein n is an integer between 0 and 300.

20. The coated substrate according to claim 1, wherein the coating layers comprise n+2 layers, comprising
a first layer and when present all odd number layers comprising cationic poly(electrolyte) or cationic poly(amino acid); and
a second layer and when present all even number layers comprising anionic poly(electrolyte) or anionic poly(amino acid),
wherein the first layer is in contact with the substrate; and
wherein n is an integer between 0 and 300.

21-22. (canceled)

23. The coated substrate according to claim 19, wherein n is an integer between 2 and 100 or between 2 and 20 or n is at least 10.

24-25. (canceled)

26. The coated substrate according to claim 1, wherein the glass comprises borosilicate glass, and wherein the ratio of the percentage of metal atoms to silicon and boron atoms in the borosilicate glass is at least 0.1.

27. The coated substrate according to claim 1, wherein the thickness of the coating is from about 1 nm to about 450 nm, or from about 3 nm to about 100 nm, or from about 3 nm to about 30 nm.

28. (canceled)

29. The coated substrate according to claim 1, wherein one or more layers of the coated substrate is a spray coated layer.

30. The coated substrate according to claim 1, wherein the substrate comprises a capillary tube or a microfluidic channel.

31. The capillary tube or microfluidic channel according to claim 30, wherein the capillary tube or microfluidic channel comprises glass.

32. The capillary tube or microfluidic channel according to claim 30, wherein the coating coats an inner surface of the capillary tube or microfluidic channel.

33. The capillary tube or microfluidic channel according to claim 32, wherein the capillary tube or microfluidic channel has an aspect ratio (length: inner diameter) of from about 2:1 to about 150:1 and/or wherein the capillary tube has an aspect ratio (length: cross sectional area) of from about 2:1 to about 191:1.

34. (canceled)

35. The capillary tube or microfluidic channel according claim 32, wherein the capillary tube or microfluidic channel has a minimum internal diameter of about 358 micrometres and a maximum internal diameter of about 2000 micrometres and/or wherein the capillary or channel has a minimum cross sectional area of about 0.1 mm² and a maximum cross sectional area of about 3.1 mm².

36. (canceled)

37. The capillary tube or microfluidic channel according to claim 32, wherein the capillary tube fills with blood to a predetermined level when held in the vertical orientation to the blood sample and wherein the predetermined level is greater than 90%, preferably greater than or equal to 95% of the capillary volume under ambient conditions.

38. The capillary tube according to claim 37, wherein the capillary fills to the predetermined level in less than about 12 seconds, preferably less than about 11 seconds.

39. The capillary tube according to claim 32, wherein the capillary tube is stored for at least 6 months or at least 9 months, or at least 12 months at ambient conditions of temperature and pressure prior to filling with blood.

40-46. (canceled)

47. The coated substrate according to claim 1 wherein the coating layers comprise an outer layer comprising anionic polyelectrolyte or anionic poly(amino acid).

48. The coated substrate according to claim 9, wherein the outer layer comprises poly(acrylic acid), poly(arginine), poly(glutamic acid) or poly(lysine).

49. The coated substrate according to claim 20, wherein n is an integer between 2 and 100 or between 2 and 20 or n is at least 10.
